(19) United States
(12) Patent Application Publication
(10) Pub. No.: US 2025/0387553 A1
(43) Pub. Date: Dec. 25, 2025

(54) DEVICE FOR QUANTITATIVELY DETERMINING THE FEED OF OXYGEN INTO BLOOD IN AN OXYGENATOR

(71) Applicant: Hamilton Medical AG, Bonaduz (CH)

(72) Inventors: Jonathan Markus Schad, Bonaduz (CH); Christoph Schranz, Maienfeld (CH); Dominik Novotni, Chur (CH); Jean-Pierre Revelly, Pully (CH)

(73) Assignee: Hamilton Medical AG, Bonaduz (CH)

(21) Appl. No.: 18/840,701

(22) PCT Filed: Jan. 30, 2023
(86) PCT No.: PCT/EP2023/052128

§ 371 Date: Aug. 22, 2024

(30) Foreign Application Priority Data
Feb. 23, 2022 (DE) 102022104340.1

(51) Int. Cl. A61M 1/36; A61M 16/00; A61M 39/22

(52) U.S. Cl. CPC A61M 1/3666; A61M 16/024; A61M 39/223; A61M 2202/0007; A61M 2202/0021; A61M 2202/0208; A61M 2202/0413; A61M 2205/3327; A61M 2205/3331; A61M 2205/3368; A61M 2205/3584; A61M 2205/3592; A61M 2205/502; A61M 2205/75; A61M 2205/7527

(57) ABSTRACT

A device for determining a feed (V′_{O2}) of oxygen into blood in an oxygenator comprises a gas flow sensor adapted to detect a flow (flow_{STPin}, flow_{STPout}) of an oxygen-containing gas mixture flowing through the oxygenator; and a gas sensor unit adapted to measure an oxygen content (p_{O2in}) of the oxygen-containing gas mixture flowing into the oxygenator and an oxygen content (p_{O2out}) of a gas mixture flowing out of the oxygenator. The device is designed to determine a discrepancy, in particular a difference, between the oxygen content (p_{O2in}) of the oxygen-containing gas mixture flowing into the oxygenator and the oxygen content (p_{O2out}) of the gas mixture flowing out of the oxygenator, and to determine the feed (V′_{O2}) of oxygen into blood flowing through the oxygenator from the difference thus determined and the flow (flow_{STPin}, flow_{STPout}) measured by the gas flow sensor.

### BACKGROUND OF THE INVENTION

The invention relates to a device for quantitatively determining a feed of oxygen into blood in an oxygenator, a device for introducing oxygen into blood in an oxygenator which is equipped with a device according to the invention for quantitatively determining the feed of oxygen, and a device for extracorporeal blood gas exchange with such a device for introducing oxygen into blood.

The invention further relates to a system for supporting the blood gas exchange of a patient by means of mechanical ventilation and extracorporeal blood gas exchange, the system comprising a device for extracorporeal blood gas exchange according to the invention and a ventilation device for mechanical ventilation of the patient's lungs.

### SUMMARY OF THE INVENTION

Physiological gas exchange comprises the introduction of oxygen into venous blood (oxygenation) and the removal of carbon dioxide from the venous blood (ventilation), so that the oxygen-poor and carbon dioxide-rich venous blood is transformed into oxygen-rich and carbon dioxide-poor arterial blood after the gas exchange has taken place. The physiological gas exchange normally takes place in the lungs. If necessary, the physiological gas exchange in the lungs can be supported by mechanical ventilation.

If the physiological gas exchange in the lungs is not sufficient to supply a patient's blood with enough oxygen, even when supported by mechanical ventilation, extracorporeal membrane oxygenation (“ECMO”) can be used in addition. In extracorporeal membrane oxygenation, oxygen is introduced into the patient's blood using an extracorporeal blood gas exchange device, which will be referred to as oxygenator in the following. Extracorporeal gas exchange also comprises removing carbon dioxide (CO₂) from the patient's blood. This is referred to as extracorporeal ventilation or extracorporeal CO₂ removal (“ECCO2R”). The combination of ECMO and ECCO2R is referred to as extracorporeal life support (“ECLS”).

In extracorporeal life support, it is desirable to be able to quantitatively determine the amount of oxygen introduced into the blood by means of extracorporeal gas exchange in order to be able to adjust the extracorporeal life support device such that a sufficient oxygen concentration is obtained in the arterial blood. It is often also desirable to be able in addition to quantitatively determine the amount of carbon dioxide (CO₂) removed from the blood in order to control that the concentration of CO₂ in the arterial blood is sufficiently reduced. It is particularly desirable to be able to coordinate the operation of the device for extracorporeal life support with the operation of an additionally operated device for mechanical ventilation in order to achieve the best possible oxygen supply, and possibly also the best possible ventilation, of the patient by means of a suitable combination of mechanical ventilation and extracorporeal life support.

It is therefore an object of the present invention to provide a device which permits quantitative determination of the extent of oxygen feed into the blood of a patient during extracorporeal membrane oxygenation. It is furthermore an object of the invention to provide a device which permits the feed of oxygen into the blood of a patient during extracorporeal membrane oxygenation and/or mechanical ventilation to be controlled in such a way that a sufficient supply of oxygen to the patient is ensured.

A device according to the invention for quantitatively determining the feed of oxygen into blood in an oxygenator (or when blood passes through an oxygenator) comprises a gas flow sensor which is designed to measure a flow of an oxygen-containing gas mixture flowing through the oxygenator; and a gas sensor unit which is designed to measure the oxygen content of the oxygen-containing gas mixture flowing into the oxygenator and the oxygen content of a gas mixture flowing out of the oxygenator. The device is designed to determine a discrepancy, in particular a difference, between the oxygen content of the oxygen-containing gas mixture flowing into the oxygenator and the oxygen content of the gas mixture flowing out of the oxygenator, and to quantitatively determine the feed of oxygen into blood flowing through the oxygenator from the difference thus determined and the flow measured by the gas flow sensor.

The flow of the gas mixture flowing through the oxygenator can be measured by means of a flow sensor, for example by means of a mass flow sensor as mass flow or by means of a volume flow sensor as volume flow.

The flow of the gas mixture flowing through the oxygenator can be measured in particular as the flow of a gas mixture flowing into the oxygenator. The fact that the oxygen content and possibly the carbon dioxide content of the gas mixture generally changes as it passes through the oxygenator can have an influence on the measured flow of the gas mixture flowing through the oxygenator. If, in addition to the feed of oxygen into the blood, carbon dioxide is also removed from the blood to approximately the same extent when passing through the oxygenator, the influence on the measured flow is so small that it can be neglected where appropriate.

The invention also comprises a method for quantitatively determining the feed of oxygen into blood in an oxygenator, the method comprising the steps of:
determining the flow of an oxygen-containing gas mixture flowing through the oxygenator, in particular the flow of a gas mixture flowing into the oxygenator and/or the flow of a gas mixture flowing out of the oxygenator;
measuring the oxygen content of the oxygen-containing gas mixture supplied to the oxygenator;
measuring the oxygen content of a gas mixture flowing out of the oxygenator;
determining a discrepancy, in particular a difference, between the measured oxygen content of the oxygen-containing gas mixture supplied to the oxygenator and the measured oxygen content of the gas mixture flowing out of the oxygenator; and
quantitatively determining the feed of oxygen into the blood flowing through the oxygenator from the previously determined difference between the measured oxygen content of the gas mixture supplied to the oxygenator and the measured oxygen content of the gas mixture flowing out of the oxygenator and from the flow of the oxygen-containing gas mixture flowing through the oxygenator.

Blood taken from a patient's blood circulation and an oxygen-containing gas mixture flow through the oxygenator at the same time. The oxygenator is designed to transfer oxygen from the oxygen-containing gas mixture into the blood. A basic idea of the invention is to determine the depletion of oxygen in the gas mixture as it flows through the oxygenator and to utilize the depletion thus determined, in particular a difference between the oxygen content of the gas mixture upstream of the oxygenator and the oxygen content of the gas mixture downstream of the oxygenator, to obtain quantitative information on the degree of oxygenation, in particular to determine the amount of oxygen that has been transferred from the gas mixture into the patient's blood in the oxygenator. The blood downstream of the oxygenator is enriched with respect to the blood upstream of the oxygenator by the amount of oxygen that has passed from the gas mixture into the blood in the oxygenator.

In corresponding manner, if desired, it is also possible to determine the enrichment of carbon dioxide in the gas mixture as it flows through the oxygenator, and from the enrichment thus determined, in particular on the basis of a difference between the carbon dioxide content of the gas mixture downstream of the oxygenator and the carbon dioxide content of the gas mixture upstream of the oxygenator (this will generally be zero), it is possible to obtain quantitative information on the degree of ventilation, in particular to determine the amount of carbon dioxide that has passed from the patient's blood into the gas mixture in the oxygenator. The blood downstream of the oxygenator is depleted in comparison to the blood upstream of the oxygenator by the amount of carbon dioxide that has been transferred from the patient's blood into the gas mixture downstream of the oxygenator.

“Upstream” of the oxygenator in the context of the invention means at, in or before the inlet of the oxygenator, “downstream” of the oxygenator in the context of the invention means at, in or after the outlet of the oxygenator, in each case in relation to the flow of the gas mixture through the oxygenator.

The oxygen content or carbon dioxide content in the gas mixture and/or in the blood can each be expressed as partial pressure of oxygen or carbon dioxide in the blood, or as partial pressure of oxygen or carbon dioxide in the gas mixture, or as mixing ratio of oxygen or carbon dioxide in the blood or in the gas mixture, or as oxygen saturation of oxygen in the blood or in the gas mixture or carbon dioxide saturation of carbon dioxide in the blood or in the gas mixture.

If the total pressure of the gas mixture is known, e.g. from an additional measurement, the concentration of oxygen or carbon dioxide in the gas mixture or the concentration of oxygen or carbon dioxide in the blood can be determined as well.

The extent of the extracorporeal blood gas exchange can be regarded in particular as the feed of oxygen into the blood when flowing through an oxygenator (degree of oxygenation) and/or the removal of carbon dioxide from the blood when flowing through the oxygenator (degree of ventilation).

A device according to the invention enables the extent of extracorporeal blood gas exchange, in particular the amount of oxygen introduced into the blood of a patient by extracorporeal blood gas exchange (i.e. the degree of oxygenation), to be determined “bloodlessly”, i.e. without intervening in the blood circulation. In this sense, the device according to the invention operates “contactlessly”. The term “contactless” in this context is intended to express that no physical contact with the blood is required to quantitatively determine the feed of oxygen into blood and/or the removal of carbon dioxide from the blood in the oxygenator.

A device according to the invention allows the gas sensor unit and/or sensors of the gas sensor unit used to measure the oxygen content in the gas mixture to be replaced without having to open and/or interrupt the blood circulation. The gas sensor unit and the sensors can therefore be replaced particularly easily and hygienically, in particular without interfering with the blood circulation.

The gas sensor unit may comprise at least one oxygen sensor which is designed to measure the oxygen content, in particular the partial pressure of oxygen, in a gas mixture flowing through the gas sensor unit.

The gas sensor unit may also comprise at least one carbon dioxide sensor (CO₂ sensor) which is designed to measure the CO₂ content, in particular the partial pressure of CO₂, in a gas mixture flowing through the gas sensor unit. By measuring the CO₂ content in the gas mixture upstream and downstream of the oxygenator, the CO₂ enrichment in the gas mixture as it flows through the oxygenator and thus the CO₂ removal from the blood (degree of ventilation) can be quantitatively determined. The extracorporeal gas exchange can be further optimized by quantitatively determining and taking into account the CO₂ removal from the blood in addition to determining and taking into account the oxygen feed into the blood.

The CO₂ sensor can be designed as a separate sensor in addition to an oxygen sensor. A combined oxygen and CO₂ sensor can also be provided which is adapted to measure both the oxygen content and the CO₂ content of the gas mixture flowing through the gas sensor unit.

Oxygen sensors, CO₂ sensors and combined oxygen and CO₂ sensors will be referred to in the following by the generic term “gas sensors”.

The device for quantitatively determining the feed of oxygen into blood in an oxygenator can be designed with only one single gas sensor such that the one gas sensor optionally measures the oxygen content and/or the CO₂ content of the gas mixture flowing into the oxygenator and the oxygen content and/or the CO₂ content of the gas mixture flowing out of the oxygenator.

In this context, a single gas sensor means that in each case only one single sensor is provided to measure the oxygen content of the gas mixture and only one single sensor is provided to measure the CO₂ content of the gas mixture. The fact that the gas sensor unit comprises only one single gas sensor can therefore mean that the gas sensor unit comprises one single oxygen sensor and one single CO₂ sensor. Alternatively, the gas sensor unit may also comprise only one single gas sensor which detects both the oxygen content of the gas mixture, optionally in the gas mixture flowing into the oxygenator and in the gas mixture flowing out of the oxygenator, or detects the CO₂ content, optionally in the gas mixture flowing into the oxygenator and in the gas mixture flowing out of the oxygenator.

By providing the same gas sensor for measuring the oxygen content and/or the CO₂ content of both the gas mixture flowing into the oxygenator and the gas mixture flowing out of the oxygenator, deviations between the respective value measured upstream and the respective value measured downstream, which are due to systematic measurement errors of the respective gas sensor, can be effectively avoided. Such measurement errors can occur between different sensors, for example due to manufacturing tolerances or due to strongly varying sensitivities between different sensors over time. When measuring with one and the same sensor, such deviations are irrelevant because they cancel each other out when determining the difference between the oxygen content or CO₂ content of the gas mixture flowing into the oxygenator and the gas mixture flowing out of the oxygenator. This greatly increases the accuracy with which such differences between the oxygen content or CO₂ content of the gas mixture flowing into the oxygenator and the gas mixture flowing out of the oxygenator can be measured, compared to measurement arrangements using two sensors, one located upstream and one downstream of the oxygenator. In addition, costs can be reduced as the costs for one oxygen sensor and the costs for a second CO₂ sensor can be saved.

A device comprising only one single gas sensor for measuring the oxygen content and, if applicable, the CO₂ content can be equipped with at least one gas switching valve which is designed to optionally supply the gas sensor with a gas mixture, in any case at least part of the gas mixture, supplied to the oxygenator, or a gas mixture, in any case at least part of the gas mixture, flowing out of the oxygenator. By switching over the at least one gas switching valve, the oxygen content in the gas mixture upstream of the oxygenator and in the gas mixture downstream of the oxygenator can thus be determined with one single oxygen sensor. Similarly, the CO₂ content in the gas mixture upstream and downstream of the oxygenator can be determined with only one single CO₂ sensor by switching over the at least one gas switching valve.

The at least one gas switching valve can be designed to switch periodically, e.g. between a first switching state in which it supplies the gas sensor at least with gas mixture supplied to the oxygenator, and a second switching state in which it supplies the gas sensor with gas mixture flowing out of the oxygenator. In this way, the oxygen content and optionally the CO₂ content of both the gas mixture flowing into the oxygenator and the oxygen content, and optionally the CO₂ content, of the gas mixture flowing out of the oxygenator can be reliably measured, also over a longer period of time or in the sense of permanent monitoring, if desired.

The at least one gas switching valve can designed, for example, to switch between the first switching state and the second switching state in intervals of between 30 seconds and 120 seconds, in particular in intervals of between 60 seconds and 90 seconds. However, shorter switching times are also possible.

When using a gas switching valve as described herein, the gas supply to the oxygenator can be modulated. Modulating the gas supply means that the gas supply to the oxygenator is increased by the gas flow fed through the gas sensor if and as long as the gas switching valve is switched to the first switching state, in order to compensate for the quantity of gas mixture that is fed past the oxygenator to the gas sensor in the first switching state of the gas switching valve.

As a result, the flow of the oxygen-containing gas mixture through the oxygenator is not significantly changed by the switching over of the gas switching valve, but in any case remains constant to such an extent that the extracorporeal gas exchange in the oxygenator is not influenced by the switching over of the gas switching valve, at least not to a relevant extent.

There can also be provided several gas switching valves that allow the entire gas flow to be passed through both the gas sensor and through the oxygenator. In this case, the previously described modulation of the gas supply, which is carried out to compensate for an amount of gas mixture that is fed to the gas sensor past the oxygenator, is not necessary.

Instead of one single gas sensor and at least one gas switching valve, the gas sensor unit may also comprise a first gas sensor and a second gas sensor, wherein the first gas sensor is arranged upstream of the oxygenator and the second gas sensor is arranged downstream of the oxygenator.

By using two gas sensors, one of which is arranged upstream and one downstream of the oxygenator, it is possible to dispense with gas switching valves as described above. By dispensing with gas switching valves, the operational safety of the device can be improved, as errors resulting from a malfunction of the gas switching valves can be ruled out.

Each of the two gas sensors upstream and downstream of the oxygenator can be designed to measure the oxygen content and/or to measure the CO₂ content in the gas mixture flowing through.

There may also be provided two gas sensors upstream and two gas sensors downstream of the oxygenator, a respective one of which is designed as an oxygen sensor for measuring the oxygen content in the gas mixture flowing through and the other one as a CO₂ sensor for measuring the CO₂ content in the gas mixture flowing through.

If at least two gas sensors are used, one of which is arranged upstream and one downstream of the oxygenator, deviations in the measured values provided by the two gas sensors at identical gas concentrations, which may result, for example, from manufacturing tolerances in the production of the gas sensors, must be sufficiently small to keep the measurement errors resulting from such deviations to a minimum.

For example, the two gas sensors can be designed such that a tolerance-related deviation between the two gas sensors is less than 0.5%, in particular less than 0.2%, for the same oxygen concentration or the same CO₂ concentration in the gas mixture. Such small deviations make it possible to measure and determine the parameters of the extracorporeal gas exchange in the oxygenator with sufficient accuracy.

The required accuracy can be achieved, for example, with sensors comprising paramagnetic cells.

The device for quantitatively determining the feed of oxygen into blood in an oxygenator may also comprise a gas conveying device which is designed to convey a gas mixture, in any case at least part of the gas mixture flowing into the oxygenator and/or in any case at least part of the gas mixture flowing out of the oxygenator, through the at least one gas sensor unit. The gas conveying device can be arranged upstream of the gas sensor unit in order to convey gas mixture with positive pressure through the at least one gas sensor unit. The gas conveying device can also be arranged downstream of the gas sensor unit in order to convey gas mixture with a negative pressure through the at least one gas sensor unit, i.e. to draw the same through the at least one gas sensor unit. Such a gas conveying device ensures that an amount of gas mixture flows through the sensor unit that is large enough to permit determination of the oxygen content and/or the CO₂ content of the gas mixture with the required accuracy.

The device may additionally comprise a gas outflow sensor which is designed to measure the flow rate of the gas mixture flowing out of the oxygenator. By measuring and taking into account the flow quantity of the gas mixture flowing out of the oxygenator, the parameters of the extracorporeal gas exchange, in particular the feed of oxygen into the blood and/or the removal of CO₂ from the blood, can be determined with even greater accuracy.

In order to achieve the desired accuracy, the gas inflow sensor and the gas outflow sensor can be designed in particular such that a tolerance-related deviation between the measured values provided by the gas inflow sensor and the gas outflow sensor when the same quantity of fluid flows through the two sensors is less than 2%, in particular less than 1%.

The device may additionally comprise at least one temperature sensor which is designed to measure the temperature of the gas mixture upstream of the oxygenator and/or the temperature of the gas mixture downstream of the oxygenator and to take the same into account in the quantitative determination of the oxygen content in the blood.

The measured temperature of the gas mixture can be used in particular to determine the humidity contained in the gas mixture.

Since water (vapor) from the blood is also transferred into the gas mixture in the oxygenator, the gas mixture flowing out of the oxygenator contains a higher proportion of humidity or water vapor than the gas mixture that has flowed into the oxygenator.

If the flow sensor and the gas sensor are arranged so close to each other in a gas supply line upstream of the oxygenator or in a gas discharge line downstream of the oxygenator that the flow sensor and the gas sensor measure the same gas mixture with the same humidity and the same temperature, the influences of the humidity on the measurement compensate each other so that the oxygen feed from the gas mixture into the blood can be determined with good accuracy without additional corrections.

In this case, an increase in flow due to increased humidity of the gas mixture downstream of the oxygenator is compensated for by a corresponding reduction in the oxygen partial pressure in the gas mixture exiting the oxygenator.

However, if the flow sensor and the gas sensor are so far apart that it cannot be guaranteed that the gas mixture flowing through the two sensors has the same humidity and the same temperature in both sensors, the values measured by the sensors must be corrected to the same gas conditions (temperature and humidity) so that the measurement results of the two sensors match.

If a “dry” gas mixture is supplied to the oxygenator, it is not necessary to correct the values measured upstream of the oxygenator. In the context of the present invention, a gas mixture is considered “dry” if it has a relative humidity of less than 2%, in particular a relative humidity of less than 1%.

Details of corrections to the values measured by the sensors and the values determined from the measured values, which are necessary in the oxygenator due to the humidity contained in the gas mixture, will be described below in connection with the embodiments shown in FIGS. 2 and 3.

A high humidity or a high water vapor content of the gas mixture flowing out of the oxygenator can lead to the deposition of water droplets or to condensation of water vapor in the components downstream of the oxygenator, in particular in the gas outflow sensor. Such humidification due to deposition or accumulation of water droplets or condensation of water vapor is undesirable, as it can impair the function of the gas outflow sensor and thus falsify the measurement results. Therefore, a heating device may be provided downstream of the oxygenator, in particular at or in the gas outflow sensor, which enables the gas mixture flowing out of the oxygenator and/or elements of the gas outflow sensor to be heated in order to reduce the humidity below the dew point so that condensation of water vapor in the gas mixture and deposition of water droplets on components are prevented.

The apparatus for quantitatively determining the feed of oxygen into blood in an oxygenator may comprise at least one releasable fluid connection enabling an oxygenator to be connected to the device such that a gas mixture flowing through the oxygenator flows through the device in such a manner that the oxygen content and/or the CO₂ content of the gas mixture upstream of the oxygenator and the oxygen content and/or the CO₂ content of the gas mixture downstream of the oxygenator is measurable by the at least one gas sensor unit of the device. Such a fluid connection permits simple and rapid replacement of the oxygenator when this is necessary or desired, for example because the oxygenator has reached its service life or the device is to be used for another patient. The oxygenator is therefore not an integral component part of a device according to the invention for quantitatively determining the feed of oxygen into blood by an oxygenator. Rather, the oxygenator is a separate exchange part which can be fluidly connected to the device, in particular by means of at least one releasable fluid connection, and can be released or detached from the device again.

The components of the device for quantitatively determining the feed of oxygen into blood by an oxygenator may be accommodated in a common housing. The common housing can be designed such that the oxygenator is also accommodated in the common housing when it is in fluid communication with the device, for example by at least one releasable fluid connection.

At least part or a section of a gas discharge line, which fluidly connects a gas outlet of the oxygenator to the gas sensor unit, may be formed with at least one moisture-permeable element which allows an exchange of moisture between the gas mixture flowing through the respective moisture-permeable element and the environment. This enables the gas mixture to release moisture into the environment and/or absorb moisture from the environment.

In particular, a moisture-permeable element can be arranged in the direction of flow of the gas mixture directly upstream of the gas outflow sensor or directly upstream of the gas switching valve in the gas discharge line.

The at least one moisture-permeable element may comprise at least one polymer membrane. In particular, the moisture-permeable element may comprise at least one “Nafion tube”.

The device can also be designed to determine the oxygen content of the blood flowing into the oxygenator from the previously quantitatively determined feed of oxygen into the blood. In particular, this can be done under the assumption that the blood leaving the oxygenator is completely saturated with oxygen, i.e. that it has an oxygen content of practically 100% of the maximum possible oxygen content. The oxygen content of the blood flowing into the oxygenator then results from the difference between 100% and the feed of oxygen into the blood in the oxygenator determined by means of the device according to the invention.

The device may have a display device which is designed to display at least one of the values measured by the sensors and/or at least one of the values determined from the measured values, in particular a value which quantitatively describes the feed of oxygen into the blood as it flows through the oxygenator, and/or a value which quantitatively describes the removal of CO₂ from the blood as it flows through the oxygenator. Displaying such values allows an operator to change parameters of the extracorporeal gas exchange, for example the flow quantity of the oxygen-containing gas mixture, the flow quantity of the blood and/or the oxygen content of the oxygen-containing gas mixture, in order to match the extracorporeal gas exchange to the current ventilation state of the patient.

The invention also comprises a device for introducing oxygen into blood, wherein the device comprises an oxygenator which is designed to be flowed through by both a patient's blood and by an oxygen-containing gas mixture in such a way that oxygen is transferred from the oxygen-containing gas mixture into the blood (oxygenation). The device for introducing oxygen into blood further comprises a device according to the invention for quantitatively determining the feed of oxygen into the blood in the oxygenator.

In addition, the device can also be provided for removing carbon dioxide from blood (ventilation). The oxygenator can also be designed to transfer carbon dioxide from the blood flowing through the oxygenator into the gas mixture flowing through the oxygenator. The device for removing carbon dioxide from blood then comprises a device according to the invention for quantitatively determining the removal of carbon dioxide from blood in the oxygenator.

The operating parameters of such a device can be set on the basis of the quantitatively determined feed of oxygen into the blood and/or on the basis of the quantitatively determined removal of carbon dioxide from the blood in such a way that the best possible extracorporeal gas exchange (in terms of oxygenation and/or ventilation) is achieved in the oxygenator and blood with a predetermined oxygen content and/or a predetermined CO₂ content can be made available to the patient.

The device may comprise a device designed to provide an oxygen-containing gas mixture, in particular an oxygen-rich gas mixture, which is supplied to the oxygenator.

Alternatively, the device may be connectable to an external device that provides an oxygen-containing gas mixture. The external device for providing an oxygen-containing gas mixture can be a blender. The external device may also be a ventilation device, which in itself is intended for mechanical ventilation by supplying ventilation gas to the lungs of a patient. In this case, part of the ventilation gas of the ventilation device can be supplied to the device according to the invention as an oxygen-containing gas mixture. If a ventilation device is used to provide the oxygen-containing gas mixture, an additional blender can be dispensed with as appropriate.

The ventilation device can also be operated in a “blender mode”, in which it is not used to ventilate the patient, but exclusively to provide oxygen-containing gas mixture to a device for extracorporeal blood gas exchange. The device for extracorporeal blood gas exchange, to which the oxygen-containing gas mixture is provided, may be a device for extracorporeal blood gas exchange according to an embodiment of the invention, but may also be another device for extracorporeal blood gas exchange.

If, in this case, the patient's lungs are to be ventilated mechanically in addition to the extracorporeal blood gas exchange, a further ventilation device can be used for this ventilation.

The device for providing the oxygen-containing gas mixture can be designed such that it enables the oxygenator to be purged. Purging the oxygenator comprises allowing a purge fluid, in particular a purge gas, to flow through the oxygenator at a high purge fluid flow rate, in particular a purge fluid flow rate of at least 12 l/min, for several seconds without exceeding a predetermined maximum pressure of the gas mixture in the oxygenator. By purging the oxygenator in this way, water that has deposited in a membrane of the oxygenator during operation and impairs the efficiency of gas exchange through the membrane can be released from the membrane and removed from the oxygenator together with the purge fluid. The efficiency of the oxygenator can be (re)improved in this way. In particular, a purge gas can be used as the purge fluid; in particular, the same gas mixture can be used that flows through the oxygenator for extracorporeal oxygenation and/or ventilation during normal operation.

The invention also comprises a device for extracorporeal blood gas exchange having a device for introducing oxygen into the blood of a patient (oxygenation) according to the invention. Optionally, the extracorporeal blood gas exchange device also comprises a device for removing carbon dioxide from a patient's blood (ventilation) as described herein.

The invention moreover comprises a system for supporting the blood gas exchange of a patient by means of both mechanical ventilation and extracorporeal blood gas exchange. Such a system according to the invention comprises a device for extracorporeal blood gas exchange according to the invention and a ventilation device provided for mechanical ventilation of a patient's lungs.

Such a system allows the patient's blood gas exchange to be carried out simultaneously both via the patient's lungs, which are supported by the ventilation device, and, if necessary, additionally by extracorporeal blood gas exchange. The ventilation device and the device for extracorporeal blood gas exchange can work in coordination with each other.

Alternatively, an additional ventilation device can be used for mechanical ventilation of the patient's lungs.

Such a system in particular permits the intracorporeal blood gas exchange through the lungs supported by the ventilation device and the extracorporeal blood gas exchange to be coordinated with each other. In this manner, the patient's blood gas exchange can be adjusted particularly well to the patient's individual needs.

The system may comprise at least one display device and be designed to quantitatively determine both the feed of oxygen into the patient's blood by the lungs/mechanical ventilation and the feed of oxygen into the patient's blood by extracorporeal blood gas exchange, as determined according to the invention, and to display them together on the display device. In particular, the system can be designed to determine the ratio between the feed of oxygen into the patient's blood by the lungs/mechanical ventilation and the, according to the invention, quantitatively determined feed of oxygen into the patient's blood by the extracorporeal blood gas exchange and to display the same on a display device.

The display device can be designed as part of the ventilation device for mechanical ventilation of the patient. In this way, all values relevant to the patient's oxygen supply can be read compactly and on a single display device, in particular on a screen of the ventilation device.

Alternatively or additionally, the display device can be formed on the device for introducing oxygen into the patient's blood.

In addition to the variables relevant to oxygenation described above, the display device can also be designed to display corresponding variables relevant to ventilation, in particular to quantitatively determine the removal of carbon dioxide from the patient's blood by the lungs/mechanical ventilation and the removal of carbon dioxide from the patient's blood by extracorporeal blood gas exchange, as determined according to the invention, and to display them together on the display device. In particular, the system can be designed to determine the ratio between the removal of carbon dioxide from the patient's blood by the lungs/mechanical ventilation and the removal of carbon dioxide from the patient's blood by extracorporeal blood gas exchange, as quantitatively determined according to the invention, and to display this ratio on the display device.

The common display of several values that are representative and/or relevant for the patient's blood gas exchange makes it easier for an operator of the system to visually detect the current status of the patient's blood gas exchange at any time and, if necessary, to adjust parameters of the ventilation and/or the extracorporeal blood gas exchange in order to improve the blood gas exchange and/or to monitor and influence the progress of a therapy.

In a system according to the invention, the ventilation device used for mechanical ventilation of the patient can be designed and arranged to provide the required oxygen-containing gas mixture to the device for introducing oxygen into the blood. In this way, an additional device for providing the oxygen-containing gas mixture, for example a blender, can be dispensed with. This can reduce the costs of the system.

Alternatively, a first ventilation device can be used to provide the oxygen-containing gas mixture required in operation to the device for introducing oxygen into blood, and an additional, second ventilation device can be used for mechanical ventilation of the patient, so that the provision of oxygen-containing gas mixture to the device for introducing oxygen into blood and the mechanical ventilation of the patient are implemented independently of each other.

The system can also have a control unit which is designed to automatically perform mechanical support of the patient's breathing by the ventilation device on the one hand and extracorporeal blood gas exchange by the device for extracorporeal blood gas exchange on the other hand in a coordinated manner in order to support the gas exchange in the blood of a patient and adapt the same as well as possible to the patient's current ventilation state.

In particular, the control unit can be designed to perform and adapt the mechanical support of the patient's breathing by the ventilation device on the one hand and the extracorporeal blood gas exchange by the device for extracorporeal blood gas exchange on the other hand on the basis of the oxygen feed into the blood, as quantitatively determined by the device for determining the feed of oxygen into blood, and/or on the basis of the determined oxygen content in the blood.

In this way, the gas exchange effected by the system, in particular the interaction between the mechanical support of the patient's breathing by the ventilation device and the extracorporeal blood gas exchange by the device for extracorporeal blood gas exchange, can be adapted particularly efficiently and reliably to the current condition of the patient.

### BRIEF DESCRIPTION OF THE DRAWINGS

In the following, embodiments of the invention will be described in more detail with reference to the accompanying drawing figures.

FIG. 1 shows a highly simplified schematic representation of a system according to the invention for supporting the blood gas exchange of a patient.

FIG. 2 shows a highly simplified schematic representation of an embodiment of a device according to the invention for quantitatively determining the feed of oxygen into blood by an oxygenator.

FIG. 3 shows a highly simplified schematic representation of an alternative embodiment of a device according to the invention for quantitatively determining the feed of oxygen into blood by an oxygenator.

FIG. 4 shows a highly simplified schematic representation of a further embodiment of a device according to the invention for quantitatively determining the feed of oxygen into blood by an oxygenator.

### DETAILED DESCRIPTION

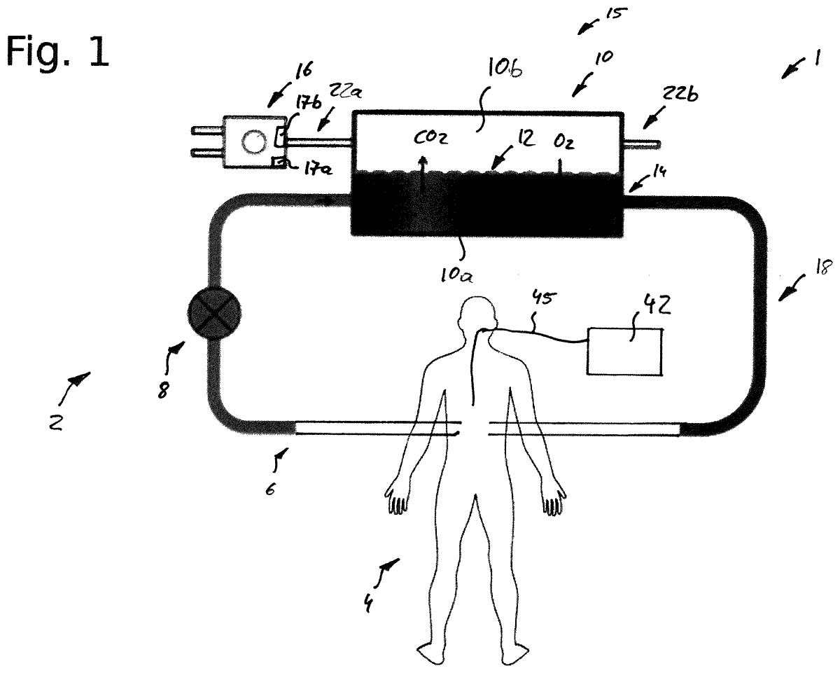

FIG. 1 shows a highly simplified schematic representation of a system 1 according to the invention for supporting the blood gas exchange of a patient 4, comprising a device 2 for extracorporeal blood gas exchange and a ventilation device 42 for mechanical ventilation of the lungs of the patient 4 through a ventilation tube 45 introduced into the lungs of the patient.

The device 2 for extracorporeal blood gas exchange comprises a blood sampling line 6, via which oxygen-depleted blood is taken from a patient 4 and supplied by a fluid conveying device 8 to a device 15 for introducing oxygen into the blood.

The device 15 for introducing oxygen into the blood comprises a so-called oxygenator 10 having a blood area 10a, shown schematically in FIG. 1 in the lower portion of the oxygenator 10, through which the blood conveyed by the fluid conveying device 8 flows, and having a gas area 10b, shown in FIG. 1 in the upper portion of the oxygenator 10, through which an oxygen-containing gas mixture flows.

The oxygen-containing gas mixture is provided via a gas supply line 22a by a device 16 for providing oxygen-containing gas mixture, for example by a blender 16 or a ventilation device 42, which is only very schematically indicated in FIG. 1. Details of the provision of the gas mixture by the blender or the ventilation device are not shown in FIG. 1 for the sake of clarity. The device 16 for providing the oxygen-containing gas mixture is equipped with pressure sensors 17a, 17b, which are designed to measure the ambient pressure p_{amb} and, if required, also the pressure p_{aw} of the oxygen-containing gas mixture provided by the device 16.

The blood area 10a and the gas area 10b of the oxygenator 10 are separated from each other by a membrane 12. The membrane 12 prevents blood from passing from the blood area 10a to the gas area 10b. However, the membrane 12 is permeable to gases, in particular to oxygen (O₂) and carbon dioxide (CO₂).

Accordingly, while the oxygen-containing gas mixture and the blood flow through the oxygenator 10, oxygen is transferred from the oxygen-containing gas mixture into the blood. At the same time, carbon dioxide is transferred from the blood to the gas mixture flowing through the oxygenator 10. After the gas mixture has flowed through the gas area 10a of the oxygenator 10, it is discharged through a gas discharge line 22b.

At the outlet 14 of the blood area 10a of the oxygenator 10, there is available blood containing oxygen and low in CO₂, which is supplied to the patient 4 via a blood supply line 18.

The illustration of the oxygenator 10 shown in FIG. 1 is a purely schematic, highly simplified representation which illustrates the mode of operation of the oxygenator 10. The real structure of the oxygenator 10 may differ from the highly simplified representation shown in FIG. 1. For example, the oxygenator 10 may have hollow fibers (not shown in the figures) that extend through the blood area 10a and around which the blood flows, with the oxygen-containing gas mixture flowing through the hollow fibers. The fibers are formed such that their walls act as membranes 12 which allow a gas exchange between the blood flowing around the fibers and the gas mixture flowing through the hollow fibers.

The device 16 for providing the oxygen-containing gas mixture may be designed such that it allows the oxygenator 10 to be purged. Purging the oxygenator 10 comprises passing a purge fluid, in particular a purge gas with a high fluid flow or gas flow, in particular a fluid flow or gas flow of at least 12 l/min, through the oxygenator 10 for several seconds, without exceeding a predetermined maximum pressure of the gas mixture in the oxygenator 10 in doing so. By purging the oxygenator 10 in this way, water that has deposited in the membrane 12 of the oxygenator 10 and impairs the efficiency of the gas exchange through the membrane 12 can be released from the membrane 12 and purged or flushed out of the oxygenator 10 together with the gas mixture. The efficiency of the oxygenator 10 can be (re)improved in this way.

In order to be able to adjust the supply of oxygen to the patient 4 by the device 2 for introducing oxygen into the blood of the patient 4 and, if necessary, additionally by a mechanical ventilation device 42 for mechanical ventilation of the patient 4 as required, it is advantageous to be able to quantitatively determine the feed V′_{O2} of oxygen into the blood of the patient 4 and/or the removal V′_{CO2} of CO₂ from the blood of the patient 4 by the gas exchange in the oxygenator 10.

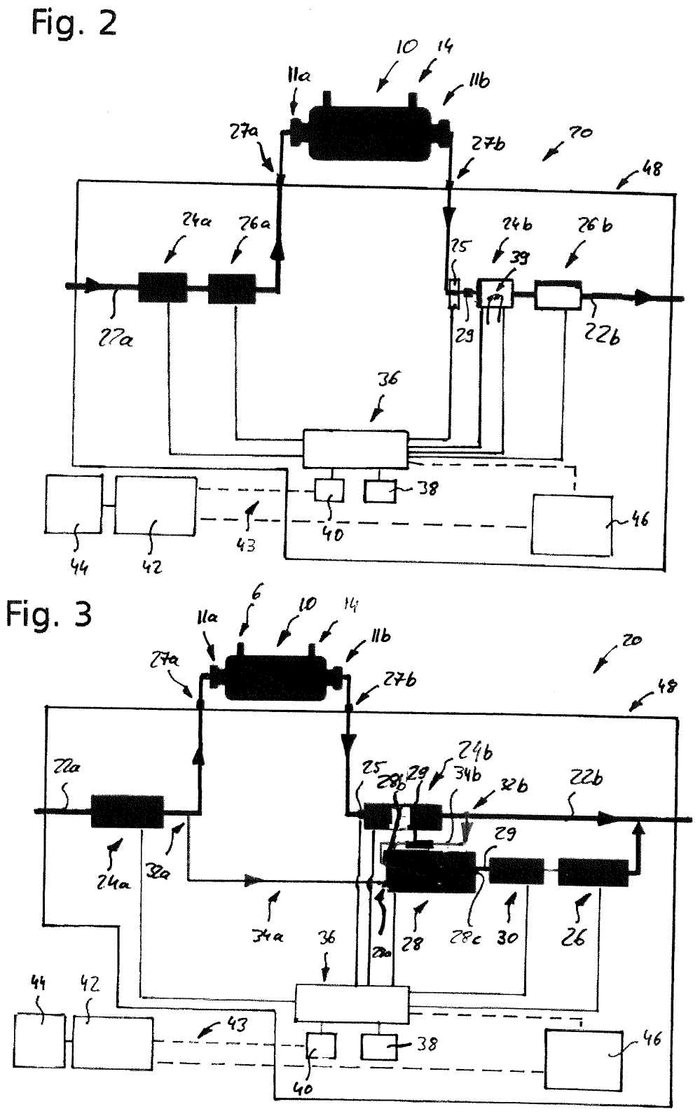

FIG. 2 shows a highly simplified schematic representation of an embodiment of a device 20 according to the invention for quantitatively determining the feed V′_{O2} of oxygen into blood by an oxygenator 10. Optionally, the device 20 can also be designed for quantitatively determining the removal V′_{CO2} of CO₂ from the blood of the patient 4.

The device 20 for quantitatively determining the feed V′_{O2} of oxygen into the blood comprises a gas inflow sensor 24a arranged in the gas supply line 22a, which is designed to measure the gas inflow flow_{STPin} (in particular a mass flow or a volume flow) into the oxygenator 10, and a first gas sensor 26a which is also arranged in the gas supply line 22a and comprises in particular an oxygen sensor 26a in order to measure the oxygen content, in particular the partial pressure p_{O2in} of the oxygen in the oxygen-containing gas mixture flowing into the oxygenator 10.

A gas outflow sensor 24b and a second gas sensor 26b are provided in the gas discharge line 22b downstream of the oxygenator 10. The gas outflow sensor 24b is provided and designed to measure the gas outflow flow_{STPout} (in particular a mass flow or a volume flow) from the oxygenator 10.

A temperature sensor can be provided in each of the gas inflow sensor 24a and the gas outflow sensor 24b, which is designed to measure the temperature of the sensor housing and the ambient temperature, respectively.

In particular, the second gas sensor 26b comprises an oxygen sensor 26b, which allows measurement of the oxygen content, in particular the partial pressure P_{O2out} of the oxygen in the gas mixture flowing out of the oxygenator 10.

Since the gas mixture flows through the oxygenator 10 always in the same direction (from left to right in the illustration of FIG. 2), the gas inflow sensor 24a and the gas outflow sensor 24b are designed such that they are in any case suitable for unidirectional continuous operation.

The gas inflow and gas outflow sensors 24a, 24b can be initially calibrated by first connecting them directly to the outlet of the device 16 for providing oxygen-containing gas mixture and flowing the oxygen-containing gas mixture through them at a defined flow rate.

The gas inflow sensor 24a can also be integrated into the device 16 for providing the oxygen-containing gas mixture. In particular, electric blenders can be equipped with a flow sensor that takes over the function of the gas inflow sensor 24a.

In addition to the oxygen sensors 26a, 26b, further (gas) sensors, in particular CO₂ sensors, can also be provided in the gas supply line 22a and in the gas discharge line 22b. There can also be provided combined gas sensors 26a, 26b, which are capable of measuring both the oxygen content p_{O2in} and p_{O2out} and the CO₂ content P_{CO2in} and p_{CO2out} in the gas mixture flowing into the oxygenator 10 and/or in the gas mixture flowing out of the oxygenator 10. In this way, in addition to the feed V′_{O2} of oxygen into the blood as it passes through the oxygenator, the removal V′_{CO2} of CO₂ from the blood can also be quantitatively determined.

If the CO₂ content in the gas mixture flowing out of the oxygenator 10 is high, it may be necessary to correct the measured gas outflow flow_{STPout} as a function of the CO₂ content, for example with a correction factor k specified by the manufacturer of the gas outflow sensor 24b:

$${flow}_{{STP}_{Out , {CorrCO}_{2}}} = {flow}_{STPOut} * ( 1 - k \frac{pCO 2}{pAmb} )$$

Alternatively, multidimensional models for the flow correction of specific flow sensors are conceivable as well. In addition to the CO₂ content, such models can also take into account the oxygen content and nitrogen content, for example. A releasable fluid connection 27a, 27b is formed between the gas supply line 22a and a gas inlet 11a of the oxygenator 10 and between a gas outlet 11b of the oxygenator 10 and the gas discharge line 22b, which allows the oxygenator 10 to be releasably connected to the gas supply line 22a and the gas discharge line 22b, such that the oxygenator 10 can be easily replaced by releasing the fluid connections 27a, 27b. Accordingly, the oxygenator 10 is not an integral component of a device 20 according to the invention for quantitatively determining the feed V′_{O2} of oxygen into blood. Rather, the oxygenator 10 is an exchange part which can be releasably connected to such a device 20 by the fluid connections 27a, 27b and is connected thereto during operation, as shown in FIG. 1. The device according to the invention is designed for coupling with oxygenators 10 of various designs, without the internal configuration of the respective oxygenator being important.

The oxygen-containing gas mixture supplied to the oxygenator 10 is “dry”, i.e. it has a humidity content of less than 2%, in particular a humidity content of less than 1%.

By determining the flow of the oxygen-containing dry gas mixture flow_{STPin} (e.g. as mass flow or volume flow) flowing into the oxygenator 10 and the content of oxygen p_{O2in} (e.g. as partial pressure or mixing ratio) in this inflowing gas mixture and by determining the flow of the gas mixture flow_{STPout} (e.g. as mass flow or volume flow) flowing out of the oxygenator 10 and the content of oxygen p_{O2out} (e.g. as partial pressure or mixing ratio) in this outflowing gas mixture, it is possible to quantitatively determine the amount of oxygen V′O2 that has been transferred from the oxygen-containing gas mixture into the blood in the oxygenator 10:

$${V}^{′} O 2 = ( \frac{p O {2}_{in}}{{p}_{AWin} + {p}_{amb}} * {flow}_{STPin} ) - ( \frac{p O {2}_{out}}{{p}_{AWout} + {p}_{amb}} * {flow}_{STPout} )$$

wherein flow_{STPout} is the previously calculated corrected flow flow_{STPout_corrCO}.

In the embodiment shown in FIG. 2, P_{AWin} can be significantly greater than p_{AWout}, especially if the gas mixture flowing through the oxygenator 10 circulates in a closed system. For example, p_{AWin} can be between about 0.5 mbar and 15 mbar, whereas p_{AWout} is virtually 0 mbar. The changes in p_{AWin} depend on the properties of the oxygenator 10 and the flow provided by the device 16.

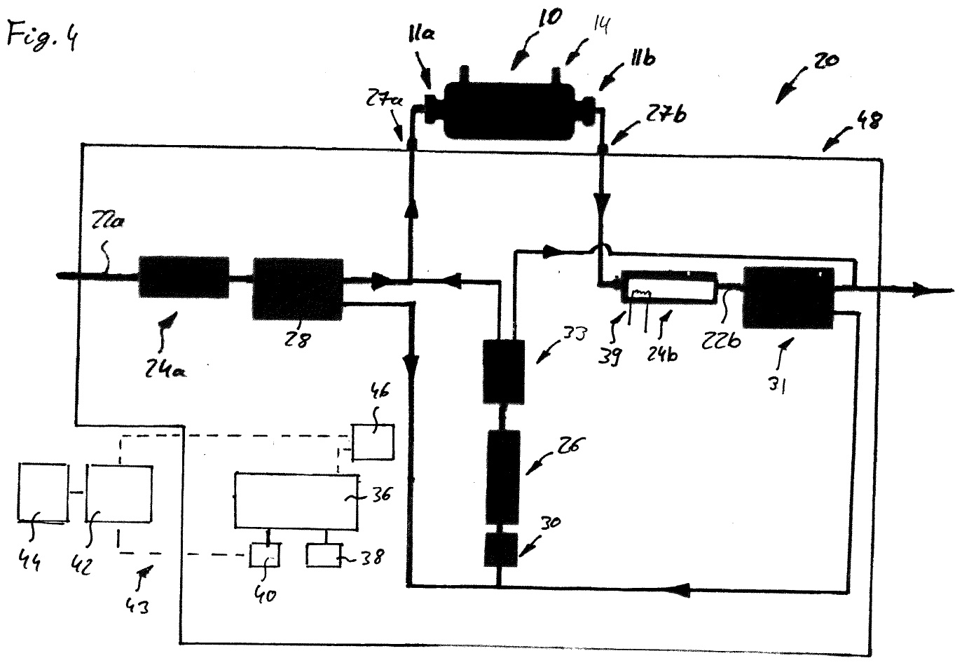

In the second embodiment, shown in FIG. 3 and described further below, the system is “open”: The gas mixture flowing out of the oxygenator 10 is discharged into the environment through the gas discharge line 22b. In an open system, p_{AWout}=p_{AWin}=0, so that p_{AWout} and p_{AWin} need not be taken into account in the calculation of V′_{O2}.

The quantity flow_{STPout} of the outflowing gas mixture, if applicable, may be the gas outflow flow_{STPout_corr} that is corrected due to a high CO₂ content, as described above.

For the measurements described here and the resulting quantitative determination of the amount of oxygen that has been transferred from the oxygen-containing gas mixture into the blood in the oxygenator 10, it is not necessary to intervene in the extracorporeal blood circulation. Therefore, for example, the gas sensors 24a, 26a, 24b, 26b, which as a rule have a limited service life, can be replaced if necessary without interrupting the extracorporeal blood circulation. Replacing the sensors 24a, 26a, 24b, 26b can therefore be carried out particularly easily and hygienically without releasing blood into the environment. Since all the variables to be measured only concern the gas mixture, the measurements do not require any physical contact with the blood. This permits the use of sensors with a simpler design (in particular gas sensors designed for measurement in gaseous fluids), which moreover have a longer service life and durability than sensors that come into contact with blood.

As the blood and gas mixture flow through the oxygenator 10, humidity is usually also transferred from the blood to the gas mixture, such that the humidity of the gas mixture is increased as it flows through the oxygenator.

Increased humidity of the gas mixture can be taken into account when determining the flow rate and the oxygen concentration of the gas mixture flowing out of the oxygenator 10. This can be achieved by positioning the sensors 24b and 26b close to each other and thereby measuring in the same gas conditions. In particular, the same conditions should prevail for both sensors with regard to the temperature and humidity content of the gas mixture.

A high humidity in the gas mixture flowing out of the oxygenator 10 can lead to condensation of water vapor contained in the gas mixture in the gas discharge line 22b and/or in the gas discharge sensor 24b. In particular, condensation of water vapor in the gas outflow sensor 24b would falsify the measurement results.

The gas outflow sensor 24b can therefore be provided with a heating device 39 that makes it possible to heat the gas outflow sensor 24b, in particular components of the gas outflow sensor 24b that come into contact with the moist gas mixture, in order to prevent water vapor contained in the gas mixture from condensing in the gas outflow sensor 24b and falsifying the measurement results. The same applies to humidity that has already condensed upstream, is transported in the form of water droplets to the gas outflow sensor 24b and condenses there. This condensed humidity can also be evaporated by appropriate heating.

Alternatively or additionally, there can also be other measures taken to prevent the condensation of humidity from the gas mixture and the accumulation of condensed water in the gas discharge line 22b and in the gas discharge sensor 24b. For example, moisture-permeable elements 29, such as moisture-permeable tubes or hoses, may be used for at least parts or sections of the gas discharge line 22b, which allow an exchange of moisture between the gas mixture flowing through the respective moisture-permeable element 29 and the environment and which in this way allow moisture to be released from the gas mixture to the environment and/or moisture to be absorbed from the environment into the gas mixture. In particular, the gas discharge line 22b may be formed with a moisture-permeable element 29 immediately upstream of the gas discharge sensor 24b, as shown in FIG. 2.

The moisture-permeable elements 29 may in particular comprise polymer membranes. Moisture-permeable elements 29 are also known as so-called “Nafion tubes”.

In order to be able to determine the feed V′_{O2} of oxygen into the blood or the removal V′_{CO2} of CO₂ from the blood with the required accuracy, the deviation between the measured values supplied by the gas inflow sensor 24a and the gas outflow sensor 24b with the same amount of flowing fluid, which may result in particular from manufacturing tolerances of the gas inflow and gas outflow sensors 24a, 24b, must be sufficiently small. The gas inflow sensor 24a and the gas outflow sensor 24b are therefore preferably designed such that the deviation between the measured values delivered by the gas inflow sensor 24a and the gas outflow sensor 24b at the same gas flow is less than 2%, in particular less than 1%.

The deviations between the measured values delivered by the first gas sensor 26a and the measured values delivered by the second gas sensor 26b at the same oxygen or CO₂ concentration in the gas mixture must also be sufficiently small. Preferably, the deviations between the measured values delivered by the two gas sensors 26a, 26b at the same oxygen or CO₂ concentration in the gas mixture are less than 0.5%, in particular less than 0.2%. This places high demands on the accuracy of the gas sensors 26a, 26b used, which in particular may only have very small manufacturing tolerances.

To avoid measurement errors resulting from tolerance-related deviations in the measured values delivered by the two gas sensors 26a, 26b, a single gas sensor 26 can be used instead of two gas sensors 26a, 26b arranged upstream and downstream of the oxygenator 10, as shown in FIG. 2, to measure the oxygen and CO₂ content of the gas mixture flowing into the oxygenator 10 as well as the oxygen and CO₂ content of the gas mixture flowing out of the oxygenator 10.

FIG. 3 shows a simplified schematic representation of an embodiment of a device 20 according to the invention for quantitatively determining the feed V′_{O2} of oxygen into blood by an oxygenator 10, which is equipped with only one single gas sensor 26. In FIG. 3, those components corresponding to components already described in relation to the embodiment shown in FIG. 2 are designated with the same reference numerals as in FIG. 2. For explanation of these components, reference is made to the description of FIG. 2, which equally applies to these components of the embodiment shown in FIG. 3.

As in the embodiment shown in FIG. 2, the embodiment shown in FIG. 3 also comprises a gas supply line 22a for supplying an oxygen-containing gas mixture to the oxygenator 10, and a gas discharge line 22b provided for discharging the gas mixture after passing through the oxygenator 10.

As in the embodiment shown in FIG. 2, a gas inflow sensor 24a is arranged in the gas supply line 22a, and a gas outflow sensor 24b and a temperature sensor 25 are provided in and on the gas discharge line 22b, respectively.

The temperature Temp_{Out} measured by the temperature sensor 25 can be used to calculate a corrected oxygen pressure p_{O2outcorr} (see below). A dedicated temperature sensor for measuring the temperature of the gas mixture can provide more accurate measurement results than a temperature sensor integrated into the heated gas outflow sensor 24b.

A first branch 32a is formed on the gas supply line 22a between the gas inflow sensor 24a and the oxygenator 10. A first gas sensor inflow line 34a leads from the first branch 32a to a first inlet 28a of a gas switching valve 28. A second inlet 28b of the gas switching valve 28 is fluidly connected by a second gas sensor inflow line 34b to a second branch 32b which is formed downstream of the temperature sensor 25 and the gas outflow sensor 24b on the gas discharge line 22b.

An outlet 28c of the gas switching valve 28 is fluidly connected to a gas sensor 26. In this manner, gas mixture from the gas supply line 22a upstream of the oxygenator 10 or gas mixture from the gas discharge line 22b downstream of the oxygenator 10 can be selectively supplied to the gas sensor 26 by switching over the gas switching valve 28.

At least one of the moisture-permeable elements 29 already described in connection with the embodiment shown in FIG. 2 may be provided in the gas sensor inflow line 34b and/or downstream of the gas switching valve 28, which enable moisture from the gas mixture exiting the oxygenator 10 to be released into the environment. In this way, moisture that has been introduced from the blood into the gas mixture in the oxygenator 10 can be prevented from condensing in the gas sensor inflow line 34b, in the gas switching valve 28 and/or in the gas sensor 26.

A gas conveying device (pumping device) 30 is provided between the outlet 28c of the gas switching valve 28 and the gas sensor 26, which is designed to convey the gas mixture through the gas sensor 26 at a positive pressure. In an alternative embodiment, which is not explicitly shown in the figures, the gas conveying device 30 may also be arranged downstream of the gas sensor 26, such that it draws or sucks the gas mixture through the gas sensor 26.

As with the gas sensors 26a, 26b of the embodiment shown in FIG. 2, the single gas sensor 26 may comprise an oxygen sensor, a CO₂ sensor or a combination of an oxygen sensor and a CO₂ sensor, such that the gas sensor 26 is capable of measuring the oxygen content p_{O2} and/or the CO₂ content p_{CO2} in the gas mixture flowing through the gas sensor 26.

In particular, the gas switching valve 28 can be designed to switch between a first switching state and a second switching state.

In the first switching state of the gas switching valve 28, part of the gas mixture flowing through the gas supply line 22a is supplied to the gas sensor 26, so that the oxygen content and/or the CO₂ content of the gas mixture flowing into the oxygenator 10 is measured by the gas sensor 26.

In the second switching state of the gas switching valve 28, part of the gas mixture from the gas discharge line 22b, which has flowed out of the oxygenator 10, is supplied to the gas sensor 26, so that the oxygen content and/or the CO₂ content of the gas mixture flowing out of the oxygenator 10 is measured by the gas sensor 26.

By measuring the oxygen content and/or the CO₂ content of the gas mixture flowing into the oxygenator 10 and the oxygen content and/or the CO₂ content of the gas mixture flowing out of the oxygenator 10 with the same gas sensor 26 in the embodiment shown in FIG. 3, deviations and measurement errors which can result from the fact that the oxygen or CO₂ content of the gas mixture flowing into the oxygenator 10 and the gas mixture flowing out of the oxygenator 10 are measured with different gas sensors 26a, 26b can be reliably avoided. In particular, deviating measurement results that are attributable to systematic errors between different sensors (such as manufacturing-related tolerances, drift processes, changes in sensitivity or sensitivity of a sensor over time) are canceled out if differences are formed between measured values that have been measured by the same sensor.

As in the embodiment of FIG. 2, the humidity of the gas mixture flowing out of the oxygenator is increased, in particular if the gas mixture supplied to the oxygenator 10 through the gas supply line 22a, for example from a blender, is dry, i.e. if the gas mixture supplied to the oxygenator 10 has a moisture content of less than 2%, in particular a moisture content of less than 1%. Such a low moisture content can be present, for example, if the gas mixture supplied to the blender originates from a high-pressure gas source. In the case of a dry gas mixture, it can be assumed that flowᵢₙ and pO2ᵢₙ are measured under the same conditions, also when the gas inflow sensor 24a and the gas sensor 26 are not arranged directly next to each other.

For the gas mixture flowing out of the oxygenator 10, it cannot be assumed that it is a dry gas mixture, since moisture from the blood is introduced into the gas mixture in the oxygenator 10, which can condense in the flow path between the gas outflow sensor 24b and the gas sensor 26.

In particular, the gas mixture arriving at the gas sensor 26, which has exited the oxygenator 10, can cool down if the line lengths are sufficiently long. As a result, the moisture contained in the gas mixture can condense, which leads to a reduction of the moisture in the gas mixture itself. As a result, the two sensors 24b, 26 do not measure the flow flowₒᵤₜ and the pressure pₒᵤₜ under the same measurement conditions.

Depending on the line length and the design of the gas sensor inflow line 34b, it may therefore be necessary to correct the result of the measurement of the gas sensor 26 when measuring the gas mixture flowing out of the oxygenator 10 in order to bring the sensor signals on the output side to the same measurement conditions.

A correction factor rp is determined for the correction as part of a calibration, which is described below. Using the correction factor rp, a corrected oxygen partial pressure pO2_{outCorr} can be calculated from the oxygen partial pressure pO2ₒᵤₜ measured by the gas sensor 26:

$$p O {2}_{outCorr} = p O {2}_{out} + rp$$

This corrects the oxygen partial pressure pO2ₒᵤₜ to the conditions at the gas flow sensor 24b. The amount of oxygen V′O2 transferred from the gas mixture into the blood is then calculated using the oxygen partial pressure pO2_{outCorr} corrected in this way:

$${V}^{′} O 2 = {flow}_{STP \_ in} * \frac{p O {2}_{in}}{{p}_{ambient}} - {flow}_{STP \_ out} * \frac{p O {2}_{outCorr}}{{p}_{ambient}}$$

Since the embodiment shown in FIG. 3 is an open system in which the gas mixture flowing out of the oxygenator 10 is discharged into the environment through the gas discharge line 22b and the ambient pressure p_{ambient} prevails in the environment, in this case p_{AWout}=p_{AWin}=0. Therefore, p_{AWout} and p_{AWin} are not included in the formula for V′O2 given here.

In the case of a closed system in which the gas discharge line 22b is not in direct fluid communication with the environment, p_{AWout} and p_{AWin} must be taken into account as described above.

For calibration, i.e. for determining the correction factor rp, dry pure oxygen gas, i.e. oxygen gas with a moisture content of less than 2%, in particular with a moisture content of less than 1%, and with an oxygen content of almost 100%, is introduced into the oxygenator 10 and humidified in the oxygenator 10 to a moisture content of 97%. In this process, the oxygenator 10 is operated such that no exchange of oxygen and/or CO₂ takes place in the oxygenator 10, whereby the oxygen content of the gas flowing through the oxygenator 10 remains unchanged at nearly 100%.

The pressure pO2_{in(dry)} of the oxygen gas flowing into the oxygenator 10 and the pressure pO2ₒᵤₜ of the dry oxygen gas flowing out of the oxygenator 10 are measured, and the correction factor rp is determined from the difference between the two pressures:

$$rp = pO {2}_{i n ( dry )} - pO {2}_{out}$$

The correction factor rp can be used to calculate a corrected oxygen pressure pO2_{outcorr}:

$${pO2}_{outcorr} = {pO2}_{out ( measured )} - {pH2O}_{out ( at 97 \% humdity )} + rp .$$

pH2Oₒᵤₜ is determined using the so-called Tetens equation, assuming that the gas mixture at the gas outflow sensor 24b has a moisture content of 97%:

$$p H 2 {O}_{out} = \frac{rh *}{100} * 6.1078 mbar * {e}^{( \frac{17.27 * {Temp}_{Out}}{{Temp}_{Out} + 237.29 ° C .} )}$$

The temperature Temp_{Out} is given in ° C. and the pressure pH2Oₒᵤₜ in mbar. With an assumed humidity of the gas at the gas outflow sensor 24b of 97%, rh*=97; i.e., the value of the humidity in percent is used in the Tetens equation for rh*.

The values determined in this way for the correction factor rp and pH2Oₒᵤₜ are stored and can be used when using the device on a patient 4 to calculate the transfer V′O2 of oxygen from the gas mixture into the blood, taking into account the changed moisture content of the gas mixture.

For the calibration described above, it is necessary to operate the oxygenator 10 such that only moisture, but no gases, in particular no oxygen and no CO₂, are exchanged in the oxygenator 10. Such operation of the oxygenator 10 can as a rule only be realized at the manufacturer's premises, in a so-called bench setup. The calibration described above can therefore generally only be carried out at the manufacturer's, not at the user's.

Devices 20 that are equipped with at least one moisture-permeable element 29 downstream of the oxygenator 10 can also be calibrated at the user's premises.

For this purpose, in a device 20 as shown schematically in FIG. 3, dry pure oxygen gas is first supplied to the gas sensor 26 through the first gas sensor inflow line 34a and the correspondingly switched gas switching valve 28, as described above. The pressure pO2_{outdry} of the dry oxygen gas is measured by the gas sensor 26 and stored for subsequent use.

In a second step, the dry pure oxygen gas is fed to the gas sensor 26 directly through the moisture-permeable element 29 and the gas switching valve 28.

In particular, the oxygen gas absorbs such an amount of moisture from the environment that the oxygen gas exiting the moisture-permeable element 29 and entering the gas sensor 26 has the same moisture content as the ambient air. The pressure pO2_{outambient} of the oxygen gas humidified in this way is measured by the gas sensor 26.

As described above, the pressure pO2ₒᵤₜ of the gas flowing out of the oxygenator 10 measured during operation on the patient 4 can then be corrected to pO2_{outcorr} using the pressure pH2O at 97% humidity, which is determined using the Tetens equation as described above, in order to determine the correct value for the amount of oxygen V′O2 introduced into the blood from the oxygen-containing gas.

Since this method, in which the humidification of the gas takes place only in the moisture-permeable element 29, does not require controlled humidification of the gas in the oxygenator 10, for which a special measuring setup is required, devices 20 equipped with at least one moisture-permeable element 29 can also be calibrated at the user's premises.

The calibration of such devices 20 can be repeated as necessary, for example after replacing one or more components of the device 20 and/or at predetermined time intervals, in order to ensure the correctness of the measurement results provided by the device 20 over as long a period as possible.

In the embodiment shown in FIG. 3, which is provided with only one single gas sensor 26, the gas supply from the device 16 for providing oxygen-containing gas mixture may be modulated to compensate for the lack of flow of oxygen-containing gas mixture that does not flow into the oxygenator 10 as it is diverted into the gas sensor 26 by the first gas sensor inflow line 34a and the gas switching valve 28. This means that the gas supply can be increased by the gas flow fed through the gas sensor when the gas switching valve 28 is switched to the first switching state, so that the flow of oxygen-containing gas mixture through the oxygenator 10 is not changed by the switching over of the gas switching valve 28, but remains substantially constant. In this way, it can be avoided that the gas exchange in the oxygenator 10 is influenced by switching over of the gas switching valve 28.

FIG. 4 shows a simplified schematic representation of a further embodiment of a device 20 according to the invention for quantitatively determining the feed V′_{O2} of oxygen into blood by an oxygenator 10. The device 20 shown in FIG. 4 is also equipped with only one single gas sensor 26, like the device 20 shown in FIG. 3.

Differently from the device 20 shown in FIG. 3, the device 20 shown in FIG. 4 comprises three switching valves 28, 31, 33. In FIG. 4, those components of the device 20 which correspond to components that have already been described with reference to the embodiments shown in FIGS. 2 and 3 are denoted with the same reference numerals as in FIGS. 2 and 3. For further explanation of these components, reference is made to the description of the embodiments shown in FIGS. 2 and 3, which also applies to the embodiment shown in FIG. 4 as regards the common components.

The embodiment shown in FIG. 4 comprises a first gas switching valve 28 which enables the oxygen-containing gas mixture flowing through the gas supply line 22a, after it has passed the gas supply sensor 24a, to be selectively supplied directly to the oxygenator 10, or first to the gas sensor 26. The gas mixture supplied to the gas sensor 26, after it has flowed through the gas conveying device 30 and the gas sensor 26, is supplied to the gas inlet 11a of the oxygenator 10 through a second gas switching valve 33.

A third gas switching valve 31 is provided in the gas discharge line 22b downstream of the gas discharge sensor 24b, which enables the gas mixture flowing out of the oxygenator 10 to be selectively fed to the gas sensor 26.

By appropriately switching over the switching valves 28, 31, 33, the oxygen-rich gas mixture from the gas supply line 22a, which flows into the oxygenator 10, or the oxygen-poor gas mixture from the gas discharge line 22b, which flows out of the oxygenator 10, can thus be selectively passed through the gas sensor 26 in order to be able to determine the oxygen content of the respective gas mixture by means of the gas sensor 26.

In the embodiment shown in FIG. 4, the entire gas flow passing through the gas supply line 22a is fed completely into the oxygenator 10. This can be achieved by switching the switching valves 28, 31, 33 accordingly.

In contrast to the embodiment shown in FIG. 3, the flow of the oxygen-containing gas mixture flowing through the oxygenator 10 can thus be kept constant. Modulation of the gas flow in the gas supply line 22a, as described before for the embodiment shown in FIG. 3, is therefore not necessary in the embodiment shown in FIG. 4.

In the embodiments shown in FIGS. 3 and 4, the gas switching valves 28, 31, 33 can be switched between their respective first switching state and their respective second switching state, for example, in intervals of between 30 seconds and 120 seconds, in particular in intervals of between 60 seconds and 90 seconds. However, shorter intervals are also possible.

The frequency with which the gas switching valves 28, 31, 33 are switched between their respective first switching state and their respective second switching state can also be variable.

The frequency with which the gas switching valves 28, 31, 33 are switched between their respective switching states can be reduced, for example, i.e. the switching intervals can be extended if the oxygen concentration at the inlet has proven to be constant over some time. In this case, it may be sufficient to check the oxygen concentration only after a change by the user and/or in longer intervals, e.g. in intervals of 10 minutes.

In the embodiments shown in FIGS. 2, 3 and 4, the device 20 for quantitatively determining the feed V′_{O2} of oxygen into the blood has in each case an evaluation device 36 which is designed to quantitatively determine, from the measurement values provided by the pressure sensors 17a, 17b (see FIG. 1), the gas inflow sensor 24a, the gas outflow sensor 24b, the at least one gas sensor 26, 26a, 26b and, if applicable, the temperature sensor 25, the feed of oxygen into the blood and optionally also the removal V′_{CO2} of CO₂ from the blood.

Assuming that the blood leaving the oxygenator 10 through the outlet 14 of the blood area 10a is saturated with oxygen almost completely, i.e. almost 100%, the oxygen content of the blood before entering the oxygenator 10 can also be quantitatively determined from the feed V′_{O2} of oxygen into the blood determined in this way. This information can be used to draw conclusions about the state, in particular the ventilation state, of the patient 4.

In the embodiments shown in FIGS. 3 and 4, in which the device 20 is equipped with at least one gas switching valve 28, 31, 33 and with only one single gas sensor 26, the evaluation device 36 is also designed as a control device which controls the at least one gas switching valve 28, 31, 33 such that the gas sensor 26 selectively measures the oxygen and/or CO₂ content of the gas mixture flowing into the oxygenator 10 as well as the oxygen and/or CO₂ content of the gas mixture flowing out of the oxygenator 10.

In the embodiment shown in FIG. 3, the evaluation device 36 may also be configured to modulate the gas supply from the device 16 for providing oxygen-containing gas mixture (see FIG. 1) to compensate for the partial amount of oxygen-containing gas mixture discharged upstream of the oxygenator 10 from the gas supply line 22a to the gas sensor 26, as described before.

The values measured by the sensors 17a, 17b, 24a, 24b, 25, 26, 26a, 26b and/or the values determined by the evaluation device 36 from these measured values, in particular the oxygen feed V′_{O2} into the blood and/or the CO₂ removal V′_{CO2} from the blood in the oxygenator 10, can be displayed on a display device 38, for example on an electronic screen 38 of the device 20.

Alternatively or in addition to the display device 38, a communication device 40 may be provided which is adapted to transmit at least one of the measured and/or determined values to a further device 42. For example, at least one of the measured and/or determined values may be transmitted to a ventilation device 42 that is provided to mechanically ventilate the patient 4. The communication device 40 may also be adapted to receive values from a further device 42, in particular from a ventilation device 42, so that the values provided by a further device, in particular the ventilation device 42, may also be displayed on the display device 38.

The communication device 40 can receive from the ventilation device 42, for example, information on the feed V′_{O2_lungs} of oxygen into the blood of the patient 4 by mechanical ventilation performed by the ventilation device 42.

These items of information can be displayed on the display device 38 together with the information determined by the evaluation device 36.

Optionally, the ratio R=V′_{O2_lungs}/V′_{O2} between the feed V′_{O2_lungs} of oxygen into the blood of the patient 4 by the mechanical ventilation and the feed V′_{O2} of oxygen into the blood of the patient by the extracorporeal blood gas exchange can also be determined and displayed on the display device 38.

Alternatively or additionally, these values can also be displayed on a display device 44 provided on the ventilation device 42, after the values determined by the evaluation device 36 have been transmitted by the communication device 40 to the ventilation device 42.

Due to the fact that all relevant values, in particular the feed V′_{O2_lungs} of oxygen into the blood of the patient 4 by mechanical ventilation and the feed V′_{O2} of oxygen into the blood of the patient 4 by extracorporeal blood gas exchange and/or their ratio R=V′_{O2_lungs}/V′_{O2} are displayed on a common display device 38, 44, the operation of the device 2 for extracorporeal blood gas exchange and the ventilation device 42 can be considerably simplified and improved, since all the items of information relevant to the blood gas exchange are combined in one place, such that they can be read out together and compared directly with one another.

Such a joint presentation enables an operator to coordinate the interaction between mechanical ventilation and extracorporeal blood gas exchange in order to supply the blood of the patient 4 with oxygen as well as possible.

In addition or as an alternative to the values relating to the feed V′_{O2}, V′_{O2_lungs} of oxygen into the blood of the patient 4, values relating to the removal V′_{CO2}, V′_{CO2_lungs} of CO₂ from the blood of the patient 4 by mechanical ventilation and/or by extracorporeal blood gas exchange and/or their ratio R=V′_{O2_lungs}/V′_{O2} to one another can also be determined and displayed on at least one display device 38, 44.

The measured and determined values can be transmitted between the communication device 40 and the further device/ventilation device 42 via a wire-bound data connection 43 or via a wireless data connection 43, in particular via a WLAN or Bluetooth® connection 43.

The sensors 24a, 24b, 25, 26, 26a, 26b, the evaluation device 36, the display device 38, the communication device 40, possibly provided gas switching valves 28, 31, 33 and a possibly provided gas delivery device 30 may be provided as an integral device in a common housing 48. Fluid connections 27a, 27b may be provided in or on the housing 48 to enable the device 20 for quantitatively determining the feed V′_{O2} of oxygen into blood to be fluidly connected to an oxygenator 10 to provide a device 2 for introducing oxygen into blood as schematically shown in FIG. 1.

The invention also comprises a system 1 for supporting the blood gas exchange of a patient 4 by means of mechanical ventilation and extracorporeal blood gas exchange, wherein the system 1 comprises a device 2 for extracorporeal blood gas exchange according to the invention and a ventilation device 42 for mechanical ventilation of the lungs of the patient 4, as shown in FIG. 1.

The device 2 for extracorporeal blood gas exchange and the ventilation device 42 for mechanical ventilation can be designed to communicate with each other via a data connection 43. The devices 2, 42 can in particular be designed to display the data and measured values relevant for the blood gas exchange, in particular values relating to the feed V′_{O2}, V′_{O2_lungs} of oxygen into the blood and/or the removal V′_{O2}, V′_{O2_lungs} of CO₂ from the blood of the patient 4 together on at least one display device 38, 44, as has been described above.

In such a system 1, the ventilation device 42 may be adapted to provide the device 2 for extracorporeal blood gas exchange with an oxygen-containing gas mixture which is supplied to the oxygenator 10 for introducing oxygen into the blood of the patient 4.

Such a system 1 may also comprise a common control unit 46 which is adapted to control the mechanical respiratory support by the ventilation device 42 on the one hand and the extracorporeal blood gas exchange by the device 2 for extracorporeal blood gas exchange on the other hand in a coordinated manner so that they jointly support the gas exchange with the blood circulation of the patient 4.

In particular, the control unit 46 can be designed to control the mechanical respiratory support by the ventilation device 42 on the one hand and the extracorporeal blood gas exchange by the device 2 for extracorporeal blood gas exchange on the other hand on the basis of the oxygen content in the blood of the patient 4 determined by the device 20 for quantitatively determining the oxygen content of blood. Moreover, the control unit 46 can be designed to take into account the previously determined CO₂ content in the patient's blood in controlling.

In this way, a coordinated gas exchange in the blood of patient 4, which takes place on the one hand by mechanical ventilation and on the other hand by extracorporeal blood gas exchange, can be simplified and improved.

1. A device for determining a feed (V′_{O2}) of oxygen into blood in an oxygenator, the device comprising:
a gas flow sensor which is designed to measure a flow (flow_{STPin}, flow_{STPout}) of an oxygen-containing gas mixture flowing through the oxygenator, in particular a flow (flow_{STPin}) of a gas mixture flowing into the oxygenator and/or a flow (flow_{STPout}) of a gas mixture flowing out of the oxygenator; and
a gas sensor unit which is designed to measure an oxygen content (p_{O2in}) of the oxygen-containing gas mixture flowing into the oxygenator and an oxygen content (p_{O2out}) of a gas mixture flowing out of the oxygenator;
wherein the device is designed to determine a discrepancy, in particular a difference, between the oxygen content (p_{O2in}) of the oxygen-containing gas mixture flowing into the oxygenator and the oxygen content (p_{O2out}) of the gas mixture flowing out of the oxygenator, and to determine the feed (V′_{O2}) of oxygen into blood flowing through the oxygenator from the difference thus determined and the flow (flow_{STPin}, flow_{STPout}) measured by the gas flow sensor.

2. The device according to claim 1,
wherein the gas sensor unit comprises at least one oxygen sensor; and/or
wherein the gas sensor unit comprises at least one CO₂ sensor which is formed separately from the oxygen sensor.

3. (canceled)

4. The device according to claim 1,
wherein the gas sensor unit comprises at least one combined oxygen and CO₂ sensor capable of measuring both the oxygen content (p_{O2}) and the CO₂ content (p_{CO2}) of the gas mixture.

5. The device according to claim 1,
wherein the gas sensor unit comprises only one single oxygen sensor and is designed such that the one oxygen sensor selectively measures the oxygen content (p_{O2in}) of the gas mixture flowing into the oxygenator and the oxygen content (p_{O2out}) of the gas mixture flowing out of the oxygenator; and/or
wherein the gas sensor unit comprises at least one gas switching valve which is designed to selectively supply to the single oxygen sensor of the gas sensor unit a gas mixture, in any case at least part of the gas mixture, supplied to the oxygenator, or a gas mixture, in any case at least part of the gas mixture, flowing out of the oxygenator; and/or
wherein the at least one gas switching valve is designed to alternately supply to the single oxygen sensor at least part of the gas mixture supplied to the oxygenator and at least part of the gas mixture flowing out of the oxygenator; and/or
wherein the gas switching valve is adapted to switch between a first switching state and a second switching state in intervals of between 30 seconds and 120 seconds, in particular in intervals of between 60 seconds and 90 seconds, wherein, in the first switching state, the gas switching valve supplies to the single oxygen sensor at least part of the gas mixture supplied to the oxygenator, and wherein, in the second switching state, the gas switching valve supplies to the single oxygen sensor at least part of the gas mixture flowing out of the oxygenator.

6-8. (canceled)

9. The device according to claim 4,
wherein the device comprises a single gas switching valve having a first inlet, a second inlet and an outlet,
wherein the first inlet of the gas switching valve is connected to a gas supply line upstream of the oxygenator;
wherein the second inlet of the gas switching valve is connected to a gas discharge line downstream of the oxygenator; and
wherein the outlet of the gas switching valve is connected to the oxygen sensor; or
wherein the gas sensor unit comprises three gas switching valves,
wherein a first gas switching valve is arranged in the device so as to enable an oxygen-containing gas mixture flowing through the gas supply line of the oxygenator to be selectively supplied directly to the oxygenator or first to the gas sensor;
wherein a second gas switching valve is arranged in the device so as to enable a gas mixture supplied to the gas sensor to be selectively supplied to the oxygenator after it has flowed through the gas sensor; and
wherein a third gas switching valve is arranged in the device so as to enable a gas mixture flowing out of the oxygenator to be selectively supplied to the gas sensor; and/or
wherein a first outlet of the first gas switching valve is connected to an inlet of the oxygenator, and
wherein a second outlet of the first gas switching valve is connected to an inlet of the gas sensor or to the inlet of a conveying device arranged upstream of the gas sensor; and/or
wherein an inlet of the second gas switching valve is connected to an outlet of the gas sensor; and
wherein an outlet of the second gas switching valve is connected to the inlet of the oxygenator; and/or
wherein an inlet of the third gas switching valve is connected to a gas discharge line downstream of the oxygenator; and
wherein an outlet of the third gas switching valve is connected to an inlet of the gas sensor or to the inlet of a conveying device arranged upstream of the gas sensor.

10-13. (canceled)

14. The device according to claim 1,
wherein the gas sensor unit comprises a first oxygen sensor and a second oxygen sensor, wherein the first oxygen sensor is arranged upstream of the oxygenator and wherein the second oxygen sensor is arranged downstream of the oxygenator; and/or
wherein the first oxygen sensor and the second oxygen sensor are designed such that a tolerance-related deviation between measured values supplied by the first and the second oxygen sensor at the same oxygen concentration in the gas mixture is less than 0.5%, in particular less than 0.2%.

15. (canceled)

16. The device according to claim 1,
wherein the device comprises a conveying device which is designed to convey the gas mixture with a positive pressure through the at least one gas sensor unit.

17. The device according to claim 1,
wherein the device comprises a gas inflow sensor which is designed to measure the flow (flow_{STPin}) of the gas mixture flowing into the oxygenator, and/or a gas outflow sensor which is designed to measure the flow (flow_{STPout}) of the gas mixture flowing out of the oxygenator; and/or
wherein the gas inflow sensor and the gas outflow sensor are designed such that a tolerance-related deviation between the measured values supplied by the gas inflow sensor and the gas outflow sensor at the same flow of flowing fluid is less than 2%, in particular less than 1%.

18. (canceled)

19. The device according to claim 1,
comprising at least one temperature sensor which is designed to measure the temperature (T) of the gas mixture upstream of the oxygenator and/or the temperature (T) of the gas mixture downstream of the oxygenator; and/or
wherein the device is designed to determine the humidity (pH2Oₒᵤₜ) of the gas mixture flowing into the oxygenator and/or of the gas mixture flowing out of the oxygenator and to take the same into account when determining the feed (V′_{O2}) of oxygen into the blood, wherein the device is designed in particular to determine the humidity (pH2Oₒᵤₜ) of the gas mixture on the basis of the measured temperature (T) of the gas mixture.

20. (canceled)

21. The device according to claim 1,
comprising a display device which is adapted to display at least one of the measured values and/or at least one value determined from the measured values, in particular a value describing the feed (V′_{O2}) of oxygen into blood flowing through the oxygenator.

22. The device according to claim 1,
comprising a communication device which is adapted to transmit at least one of the measured values and/or at least one value determined from the measured values, in particular a value describing the feed (V′_{O2}) of oxygen into blood flowing through the oxygenator, to another device; and/or
wherein the communication device is designed to transmit the at least one value to the other device in wire-bound or wireless manner, in particular via a WLAN or Bluetooth® connection.

23. (canceled)

24. The device according to claim 1,
wherein the device comprises at least one releasable fluid connection which enables an oxygenator to be connected to the device such that a gas mixture flowing through the oxygenator flows through the device in such a manner that the oxygen content (p_{O2in}) and/or the CO₂ content (p_{CO2in}) of the gas mixture upstream of the oxygenator and the oxygen content (p_{O2out}) and/or the CO₂ content (p_{CO2out}) of the gas mixture downstream of the oxygenator can be measured by the at least one gas sensor unit.

25. The device according to claim 1,
wherein the device is designed to determine the oxygen content of the blood flowing into the oxygenator from the determined feed (V′_{O2}) of oxygen into the blood.

26. The device according to claim 1,
wherein the components of the device are accommodated in a common housing.

27. The device according to claim 1,
wherein at least part or a section of a gas discharge line, which fluidly connects a gas outlet of the oxygenator to the gas sensor unit, is formed with at least one moisture-permeable element which allows an exchange of moisture between the gas mixture flowing through the respective moisture-permeable element and the environment; and/or
wherein a moisture-permeable element is provided in the flow direction of the gas mixture immediately upstream of the gas outflow sensor or immediately upstream of the gas switching valve in the gas discharge line; and/or
wherein the at least one moisture-permeable element comprises a polymer membrane, wherein the moisture-permeable element comprises in particular at least one “Nafion tube”.

28-29. (canceled)

30. A device for introducing oxygen into blood,
wherein the device comprises:
an oxygenator which is designed to have blood and an oxygen-containing gas mixture flowing therethrough such that oxygen from the oxygen-containing gas mixture is transferred into the blood; and
a device for quantitatively determining the feed (V′_{O2}) of oxygen into the blood according to claim 1.

31. The device for introducing oxygen into blood according to claim 30,
wherein the device comprises a device for providing oxygen-containing gas mixture, in particular oxygen-rich gas mixture; and/or
wherein the device for providing oxygen-containing gas mixture is a ventilation device or a blender.

32. (canceled)

33. Use of a ventilation device or a blender for providing an oxygen-containing gas mixture, in particular an oxygen-rich gas mixture, to a device for introducing oxygen into blood according to claim 30.

34. A device for extracorporeal blood gas exchange comprising a device for introducing oxygen into blood according to claim 30.

35. A system for supporting the blood gas exchange of a patient by means of mechanical ventilation and extracorporeal blood gas exchange, comprising:
a device for extracorporeal blood gas exchange according to claim 34; and
a ventilation device for mechanically supporting breathing by the lungs of the patient.

36. The system for supporting the blood gas exchange of a patient according to claim 35,
wherein the system is designed to quantitatively determine both the feed (V′_{O2_lungs}) of oxygen into the blood of the patient by mechanical ventilation and the feed (V′_{O2}) of oxygen into the blood of the patient by extracorporeal blood gas exchange and to display the same on a display device; and/or
wherein the system is designed to determine the ratio (R=V′_{O2_lungs}/V′_{O2}) between the feed (V′_{O2_lungs}) of oxygen into the blood of the patient by mechanical ventilation and the feed (V′_{O2}) of oxygen into the blood of the patient by extracorporeal blood gas exchange and to display said ratio on a display device; and/or
wherein the system is designed such that the ventilation device provides the oxygen-containing gas mixture to the device for introducing oxygen into blood; and/or
wherein the system comprises a control unit which is adapted to automated perform mechanical respiratory support by the ventilation device on the one hand and extracorporeal blood gas exchange by the device for extracorporeal blood gas exchange on the other hand in a coordinated manner in order to support the gas exchange with the blood circulation of the patient; and/or
wherein the control unit is adapted to perform the mechanical respiratory support by the ventilation device on the one hand and the extracorporeal blood gas exchange by the device for extracorporeal blood gas exchange on the other hand on the basis of the feed (V′_{O2}) of oxygen into the blood of a patient quantitatively determined by the device for determining the feed of oxygen into blood.

37-53. (canceled)
